(19) United States
(12) Patent Application Publication
(10) Pub. No.: US 2015/0024496 A1
(43) Pub. Date: Jan. 22, 2015

(54) Methods and Systems for Utilizing Sperm for Molecular Delivery

(71) Applicant: Elwha LLC, Bellevue, WA (US)

(72) Inventors: Roderick A. Hyde, Redmond, WA (US); Tony S. Pan, Cambridge, MA (US); Lowell L. Wood, JR., Bellevue, WA (US)

(21) Appl. No.: 13/946,156

(22) Filed: Jul. 19, 2013

(51) Int. Cl. C12N 15/85; A61B 17/435; C12N 15/82

(52) U.S. Cl. CPC C12N 15/8509; C12N 15/8202; A61B 17/435
USPC 435/449; 435/285.1; 600/34

(57) ABSTRACT

Disclosed herein include embodiments related to delivery of molecular molecules by a sperm cell to an egg cell for expression, including transient expression, in a fertilized progeny. Further embodiments relate to computerized systems for assisting in the disclosed methods.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority application(s)). In addition, the present application is related to the “Related Applications,” if any, listed below.

### PRIORITY APPLICATIONS

None.

### RELATED APPLICATIONS

United States patent application No. To be Assigned, entitled METHODS AND SYSTEMS FOR UTILIZING SPERM FOR MOLECULAR DELIVERY, naming Roderick A. Hyde, Tony S. Pan and Lowell L. Wood, Jr. as inventors, filed 19 Jul. 2013 with attorney docket no. 0712-002-003-000000, is related to the present application.

United States patent application No. To be Assigned, entitled METHODS AND SYSTEMS FOR UTILIZING SPERM FOR MOLECULAR DELIVERY, naming Roderick A. Hyde, Tony S. Pan and Lowell L. Wood, Jr. as inventors, filed 19 Jul. 2013 with attorney docket no. 0712-002-004-000000, is related to the present application.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

Utilizing sperm for delivery or transient expression of molecular payloads provides a means for delivering molecularly useful or critical factors to an egg cell or a developing zygote/embryo. In an embodiment, the molecular delivery includes at least one factor for assistance in early development of the zygote or embryo. In another embodiment, the molecular delivery includes at least one factor utilized by a healthy egg cell during fertilization. In an embodiment, the molecular delivery includes one or more transcription factors needed for embryonic genome activation. In an embodiment, pollen grains are utilized for delivery or transient expression of molecules to progeny in plants.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a partial view of an embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Utilizing sperm for delivery or transient expression of molecular payloads provides a means for delivering molecularly useful or critical factors to an egg cell or a developing zygote/embryo. In an embodiment, the molecular delivery includes at least one factor for assistance in early development of the zygote or embryo. In another embodiment, the molecular delivery includes at least one factor utilized by a healthy egg cell during fertilization. In an embodiment, the molecular delivery includes one or more transcription factors needed for embryonic genome activation (EGA). In an embodiment, pollen grains are utilized for delivery or transient expression of molecules to progeny in plants.

In an embodiment, a method comprises: selecting one or more sperm cells or precursor thereof from pollen; transducing the one or more selected sperm cells or precursor thereof with at least one DNA sequence, RNA transcript or protein; combining a transduced sperm cell or precursor thereof with an egg cell for fertilization; and receiving molecular information related to the progeny resulting from the fertilization. In an embodiment, the plant includes a food crop, or ornamental plant.

In an embodiment, receiving molecular information related to the progeny includes receiving from a third party or performing the analysis of the progeny itself. In an embodiment, the molecular information or analysis of the progeny includes testing for at least one of gene insertion, gene deletion, gene mutation, alteration in methylation, protein insertion, protein mutation, or manipulation of cell ploidy. In an embodiment, the method further includes verifying the presence of the at least one DNA sequence, RNA transcript or protein in the progeny. In an embodiment, the DNA construct is non-integrating into the genome of the sperm or precursor cell. In an embodiment, the RNA transcript is generated from a non-integrating DNA construct. In an embodiment, the RNA transcript includes at least one of microRNA, mRNA, tRNA, or siRNA.

In an embodiment, receiving molecular information related to the progeny includes receiving molecular information regarding the quality of at least one gene or gene product.

In an embodiment, the molecular information may be derived by the same user or another user of the method. In an embodiment, receiving molecular information related to the progeny includes receiving transmitted molecular information (e.g., wired or wireless transmission, including wi-fi transmission). In an embodiment, the molecular information includes one or more of information regarding genetic sequencing, RNA sequencing, or protein sequencing. In an embodiment, the molecular information is regarding the quality of at least one gene or gene product, including for example, at least one of telomere length, protein folding, gene expression, nucleotide or amino acid deletion, nucleotide or amino acid insertion, nucleotide or amino acid mutation, protein mutation, methylation status, or demethylation status.

In an embodiment, the method, device, or system includes identifying at least one desired genetic, transcription, or translation characteristic from the sequence of the progeny.

In an embodiment, a second round of receiving molecular information related to the progeny is performed subsequent to implanting the progeny into a suitable subject. In an embodiment, receiving molecular information related to the progeny is performed at least at one time point post-fertilization (e.g., pre-implantation of the progeny, or post-implantation of the progeny). In an embodiment, receiving molecular information related to the future progeny can be performed prior to fertilization (e.g., screening gametes as discussed herein).

In an embodiment, the molecular information is obtained by analysis of at least a portion of one or more cells from the progeny.

In an embodiment, a molecular payload includes a molecularly useful or critical factor needed for the first several rounds of cell division. In an embodiment, the molecular payload includes at least one of a small molecule, protein or peptide(s), transcription factor, miRNA, siRNA, or other molecule(s).

In an embodiment, the molecular payload of the sperm does not integrate into the genome of the sperm, egg, or fertilized progeny. Instead, it is only transiently expressed and/or utilized in its own form or processed form (e.g., mRNA, protein, peptide, etc.). In this way, there is no genetic alteration to the genome of the progeny, but rather only an alteration at the expression level or protein level of the gene product.

In an embodiment, the sperm cell or precursor includes at least one of a spermatogonium, spermatocyte, or spermatid. In an embodiment, the sperm cell includes pollen grains. In an embodiment, the egg cell or precursor thereof includes at least one oocyte, ootid, or oogonium. In an embodiment, the subject includes a plant or animal. In an embodiment, the at least one subject includes at least one of a plant, alga, or animal. In an embodiment, the at least one subject includes at least one of a vertebrate or invertebrate. In an embodiment, the at least one subject includes at least one of an amphibian, mammal, reptile, fish, or bird. In an embodiment, the at least one subject includes at least one human. In an embodiment, the at least one plant includes at least one of a food crop, ornamental, aquatic plant. In an embodiment, the at least one plant includes at least one flowering plant, herb, shrub, bush, tree, or vegetable.

In an embodiment, the one or more sperm cells or precursors thereof are selected for modification according to predetermined criteria, including for example, level of motility, ability to bind hyaluronic acid, high level of normal morphology, chromatin condition, storage conditions of the cell, or ability to utilize ATP. In an embodiment, the storage conditions of the cell include at least one of duration of storage time, storage temperature, storage size, reproductive cell dilution, or storage solution(s).

In an embodiment, the molecular payload includes telomerase. In this way, the embryo's telomerase is lengthened, providing an increased ability to withstand age and/or disease. However, in this embodiment, since the expression is transient, and cells are not genetically modified by insertion or deletion of genes, the effect is highly regulated. In an embodiment, a transcription factor for activating the inherent embryonic telomerase is delivered, thus increasing the length of the embryonic telomeres by delivering transcription factors. For example, transcriptional control of the telomerase catalytic subunit gene in humans, human telomerase reverse transcriptase (hTERT), may be regulated by other gene products, including oncogene products (e.g., c-Myc) tumor suppressor gene products (e.g., WT1, p53, etc.), or cell division transcription factor families (e.g., ER81, Ets, etc.). See, for example, Horikawa, et al. Carcinogenesis vol. 24, 7: 1167-1176 (2003), which is incorporated by reference.

In another example, the telomerase protein or transcript is included in the sperm payload in order to increase the telomere length in the sperm cell itself. Telomeres are required for normal chromosomal segregation and stability, as well as normal cell division. See Kumar et al., CLINICS 2013; 68(S1): 5-14, available online at creativecommons.org, the content of which is incorporated herein by reference.

In an embodiment, the protein utilized in the sperm payload includes TERC (telomerase RNA component), DKC1 (dyskeratosis congenital 1), TET1 (tet methylcytosine dioxygenase 1), DNMT1 (DNA (cytosine-5)-methyltransferase 1), DNMT2 (DNA (cytosine-5)-methyltransferase 2), or DNMT3 (DNA (cytosine-5)-methyltransferase 3).

In an embodiment, the molecular payload in the modified sperm includes one or more transcription factors usually provided by maternal transcripts (but for whatever reason, may be deficient or suspected of being deficient), that allow for the transcription of essential embryonic proteins required for zygotic or embryonic gene activation to initiate or continue once begun (e.g., elF 1A). In an embodiment, Ca2+ is carried by the sperm, which acts to increase translation of maternal mRNAs, which are sequestered by translation inhibiting complexes, whereas Ca2+ release causes phosphorylation of the inhibiting complex and release of the mRNA and enabling polyadenylation and translation to occur. Further, since maternal proteins are regulated post-translation by modification of the protein phosphorylation state, embryonic kinases and/or phosphatases may be included in the sperm's payload for delivery to the zygote and stimulation of protein activity, targeting of the protein for degradation or changing the protein's subcellular location.

For example, the maternal to zygotic transition occurs during early embryonic development (e.g., 2 cell stage in certain mammals) and includes switching from maternal transcripts to gene activation under the control of the zygotic genome. This transition requires both zygotic gene activation and degradation of maternal products (e.g., proteins, mRNA transcripts, etc.). Several factors are known to play a role in the maternal to zygotic transition, including the nucleocytoplasmic ratio of the zygote. During this early transition, the male pronucleus supports a higher level of transcription than the female pronucleus, and the overall level of transcription is significant in the late 1 cell embryo, reaching 30-40% of that observed in the late 2 cell embryo. See for example, Zeng and Schultz, Dev. Biol. (2005) 283: 40-57, which is incorporated herein by reference.

Likewise, repression of the zygotic genome makes it necessary for enhancers to relieve the repressive state by inducing histone acetylation or by inhibiting the second round of DNA replication. Chromatin remodeling that occurs during the 1 and 2 cell stages provides access to cis-cognate DNA binding sequences for transcription factors.

One of the first steps in zygotic genome activation includes overcoming the silencing of zygotic gene expression due to chromatin modifications or lack of transcription factors, among others. Several maternal transcripts that are not re-expressed later in development have been identified (e.g., H1foo and Msy2 in mammals). Id. Clearance of maternal transcripts is mediated, among other ways, by the 3′ untranslated region of the maternal transcripts themselves, the regions of which are recognized by regulatory proteins that cause destabilization or degradation of the transcripts. In addition, microRNAs play a role in degrading maternal transcripts (miR-430 or miR-427), which may be included in the payload of the modified sperm described herein. The microRNAs function to degrade maternal transcripts by deadenylation, and possibly other mechanisms. For example, miR-427 targets cell cycle regulators such as cyclin A1 and cyclin B2. See for example, Lund et al., RNA (2009) 15:2351-2363, which is incorporated herein by reference. That is, specific regions in the 3′ UTRs of maternal cyclin A1 and cyclin B2 mRNAs are necessary and sufficient for deadenylation and degradation of the maternal transcripts, independent of translation. Id. Binding of particular proteins (pumilio, Puf, CCR4-NOT, etc.) contributes to the degradation of the maternal transcripts at the recognition sites described.

In an embodiment, the payload of the sperm includes an RNA transcript (e.g., mRNA) of one or more genes related to early gene activation, including:

Atpi, Bat1, Cpd, Ddx18, Ddx21, Gdi2, Glg1, Gm2a, H2afz, Hmox2, Imp-1, Jtva, Lamp2, Mina53, Mt1a, Myc, Mycn, Ndrg1, No15a, P1s3, Psat1, Rp113, Rp17, Rp19, Rp1113, Rp119, Rp121, Rp123, Rp127, Rp130, Rp132, Rp135, Rp141, Rps13, Rps18, Rps19, Rps20, Rps23, Rps27, Rps4x, Sasdh, Sardh, Shmt1, Tde1, Apex1, Bag4, Ccnd1, Ccne1, Ccne2, Cdkn1a, Cu13, Dnaja1, Foxg1b, Hdac1, Hes1, Hesx1, Hmgb1, Hmgb2, Hspa8, Hsph1, Jarid1b, K1f4, Mta2, Nme2, Pax9, Pim1, Prkch, Procr, Prtn3, Rad9a, Rnf7, Set, Skp2, Terf1, Tle1, Tob1, Topbp1, Vh1, Znf151, Ap1p2, Bcl2a1, Bcl21l1, Bid, Casp3, Ccnt1, Ccnt2, Cdk9, Ceacam1, Cts1, Dcc, Fem1b, Gas2, Gcnt2, Gtf2f1, Gtf2f2, His1, Hla-dqb1, Hmox2, Hnrpu, Htatsf1, Irf3, Krt8, Krt18, Mcl1, MTA2, ARID4A, BCOR, MBD2, YY1, Mhc2ta, Pcaf, Ptma, Rea, Rfxank, Rfxap, Supt4h1, Supt5h, Troap, Wee1, Cd38, Clk1, Cpsf1, Ddx39, Eif1a, Eif3s4, Eif3s5, Eif3s6, Eif3s8, Eif3s10, Eif3s6ip, Eif4a2, Eif4e, Gspt1, Hmgn2, Hnrpa1, Lck, Lin-4, Let-7, Mapk13, Mknk2, Nup214, Nxf1, Pabpc1, Paip1, Prpf4b, Rbm8a, Rps25, Ryr1, Sfrs1, Sfrs5, S=1, Thoc4, Thy1, Tnpo3, U2af1, Ub15, Atf3, Bc13, Cbx3, Cdc2, Cdc6, Cdc25c, Cdkn2c, Cdkn2d, Chek2, Csnk2a1, Dub1, E2f6, Fgf1, Gata1, Gadd45a, Hdac1, Klf4, Hspa2, Junb, Lats2, Madh7, Mizf, Mta2, Mcm3, Mcm4, Mcm6, Orcl1, Paf53, Pck1, Polr1a, Polr1c, Polr1d, Pttg1, Pttg1ip, Rps10, Sei1, Smarcc1, Trim28, Ube2a, Ubtf, Zfp118, Akap8, Arih1, Arih2, Blnk, Bcos, Clqbp, Cbl, Ccne1, Ccne2, Cdc2, cdc6, cdc25c, cdk5s1, cdkn1a, Chek2, cul3, Crk1, Dck, Eif4e13, Fbl, Foxc2, Icam1, Kcnab2, Mki67ip, Msn, Nme2, Ncl, Pim1, Ppia, Pspf4b, Pttg1, Pin1, Pin4, Pip5k1a, Plec1, Ppm1a, Prkar2a, Prkca, Prkcz, Rpl10a, Rnf7, Rps10, Rps6, Set, Sfss1, Sfss5, Sfpq, Slc1a1, S1c2a4, Spry2, Tmpo, Tnpo3, Tob1, Top2a, Ube2a, Top2a, Ube213, Vi12, Wdr12, Btg1, Clns1a, Ddx20, Dhx9, Dis3, Etv3, Fbl, Gemin5, Gemin6, Gemin7, Hrmt112, Ipo7, Khdrbs1, Kpnb1, Kpnb3, Mep50, Nup153, Nxf1, Ran, Ranbp2, Rangap1, Rnut1, Rp113, Sip1, Skb1, Smndc1, Snrpb, Snrpd1, Snrpd2, Snrpd3, Snrpf, Tec, Xpo5, Xpot, Znf259, Arc, Coi1, Ddx20, Dhx9, Fbl, Fgf2, Fkbp3, Fmr1, Gan, Gemin4, Gemin5, H2-k, Lamr1, Lgals1, Lgals3, Map1b, Mdk, Ncl, Nola1, Nola2, Nola3, Nolc1, Nop5/nop58, Nufip1, Prpf8, Rp113a, Rps7, Sip1, SmN1, Snrpb, Snrpd2, Snrpd3, Snrpe, Snrpf, Tfpt, Apoa1, Apoe, Col11a2, Hspb1, Ipo9, Jrk, Mt1a, Myc, Mycn, Ndufa2, Pin4, Pltp, Polr2a, Polr2b, Polr2c, Polr2d, Polr2e, Polr2f, Polr2g, Polr2h, Polr2i, Polr2j, Polr2k, Polr21, Rp17, Rp126, Rp127a, Rps3, Rps7, Rps9, Rps11, Tbp, Xab2, Znf263, Ascl1, Asid4a, Axin1, Cbx3, Cbx8, Clock, Csnk1d, Csnk1e, Dbp, Ddc, Dio1, Dv11, Dv1111, E2f6, H-l(3)mbt-like, Hdac3, Id1, Mblr, Ngfrap1, Per1, Pgd, Ppia, Ppp2ca, Prdx6, Ring1, Ring2, Rnf134, Rrm1, Rrm2, Rybp, Senp2, Stat5b, Thrb, Yaf2, Ywhae, or Yy1. Id.

In an embodiment, the molecular payload includes at least one HDx protein involved in histone acetylase. For example, acetylation and deacetylation are regulated by enzymes and are involved in regulation of gene expression through the access to histone proteins and by working in conjunction with transcription factors, particularly TFIIIA. See U.S. Pat. No. 8,399,233, which is incorporated herein by reference.

In an embodiment, the molecular payload includes methyltransferase. In this way, DNA replication fidelity in the early embryo has improved performance. In an embodiment, delivery of methyltransferase assists in epigenetic characteristics of the early embryo. For example, modifying DNA methylation has been considered important for transcription of embryonic gene activation and subsequent embryonic development. See, for example, Silva, et al., Epigenetics, Abstract, 6(8): 987-93 (2011), which is incorporated herein by reference.

For example, in the absence of methylase related to a paternal gene, the paternal insulator is left unmethylated, which allows for the corresponding transcription factors to bind, and promotes maternal gene expression, leading to silencing of the paternal gene. See for example, Biliya and Bulla, Exp. Biol. And Med., (2010), 235: 139, which is incorporated herein by reference.

For example, at fertilization, the paternal genome exchanges protamines for histones, undergoes DNA methylation, and acquires histone modifications, whereas the maternal genome appears epigenetically more static with passive DNA demethylation and further reorganization of histone modifications. See Morgan et al., Hum. Mol. Genetics, 2005: Vol. 14, pp. R47-R58, which is incorporated herein by reference.

In an example, H3K9me3, H3K9me2, H3K4me1, H3K9me1, and H3K27me1 are markers that have been associated with methylation and DNA demethylation in the pronucleus. Id. In addition, various cytidine deaminases coupled with base excision repair are believed to assist in DNA repair mechanisms, likely in tangent with DNA repair genes in the young embryo. In an embodiment, one or more DNA repair mechanisms is bolstered by a RNA transcript or protein incorporated into a sperm cell prior to fertilization of the offspring. In an embodiment, for example, a transcript of one or more DNA repair genes that are underexpressed in a particular egg cell are included in the sperm cell for delivery during fertilization.

In another example, one or more specific transcription factors are included in the payload of the sperm for fertilization that are sufficient to activate expression one or more genes of the progeny, or of the egg cell, but that do not integrate into the genome of the progeny. For example, in order to grow plant or animal food products faster or bigger, one or more RNA transcripts or proteins are included in the sperm or pollen for delivery to the egg cell during fertilization. In an embodiment, the RNA transcript or protein is derived from another species.

In an embodiment, the sperm cell includes a factor that assists in the success of fertilization or implantation. Assisted reproductive technology has increased the pregnancy rate of otherwise infertile couples. In an embodiment, the modified sperm cells of an embodiment described herein are utilized in assisted reproductive technology for humans, or in animal husbandry to assist in attaining a desired outcome. For example, in an embodiment, the transduced sperm cells described herein are utilized in conjunction with in vitro fertilization, intracytoplasmic sperm injection, cryopreservation, or artificial (e.g., intrauterine) insemination for humans or in animal husbandry.

For example, in vitro fertilization involves combining egg and sperm cells outside a subject's body, such as in a petri dish. This may include fertilization of plant or animal cells. Once the embryo(s) form, they are placed in a subject for further growth (particularly for animal subjects) or further cultured (particularly for plants, that are able to develop to a higher stage in vitro).

In the case of intracytoplasmic sperm injection, egg cells are injected with sperm as part of an in vitro fertilization process. This process may be utilized with plant or animal cells, as well.

In the case of artificial insemination, typically sperm cells (or whole semen) is introduced into a subject (e.g., in an animal subject typically the sperm cells or whole semen is introduced into the vagina, uterus, or oviduct) for purposes of fertilization. In plants, selective pollination is included here.

In any of these instances, the sperm cells may be modified as described herein, and the modified sperm cells (e.g., transduced with an RNA transcript or protein, etc.) may be cryopreserved or otherwise frozen or preserved for future fertilization(s). In certain instances, some of the modified sperm is utilized immediately, and the rest preserved for a later time (e.g., for use with progeny of the first fertilization or for another fertilization of unrelated subject(s)). In certain instances, the entirety of the modified sperm cells is preserved for later use. In certain instances, the entirety of the modified sperm cells is utilized immediately for fertilization and none is preserved for later use.

In an embodiment, the sperm is modified to carry the payload (e.g., RNA transcript, protein, etc.) by one or more of episomal vector, liposome, electroporation, injection, or conjugation. For example, siRNA has been chemically conjugated to lipids, polymers, peptides, or inorganic nanostructural materials. See Jeong, et al., Bioconjug. Chem. (2009) 20(1), pp. 5-14, which is incorporated by reference.

For example, animal husbandry has been utilized in order to maintain a standard (i.e. more efficient) breeding schedule, as well as to improve the quality and/or quantity of progeny produced in breeding. Animal husbandry has also been utilized to increase the efficiency of the animals' ability to utilize feed for purposes of growing healthier, bigger, or stronger, as well as for potentially producing better quality or quantity of progeny.

In an embodiment, the sperm cell includes a factor (e.g., protein or RNA transcript) that induces a somaclonal variation such as a chromosomal structure, gene structure, gene product structure, gene amplification, or gene methylation in the progeny. For example, in plant tissue culture, epigenetic variations can be visualized at a morphogenic level, at a microscopic level, or at a molecular level.

In an embodiment, epigenetic factors that can be included in the payload of the modified sperm include factors involved in DNA methylation, signaling factors, histone tail modifications, targeted histone retention, and protamine incorporation into chromatin.

In an embodiment, the methods and systems described herein relate to selecting one or more egg cells for fertilization with the modified sperm cell(s). In an embodiment, the egg cell(s) are selected as determined by several criteria, including for example, screening one or more egg cells based on age of the donor, presence of at least one genetic marker, overall morphology, or chromosomal condition, or expression of one or more genetic markers, such as SPSB2 or TP5313 gene.

In an embodiment, a computer-implemented method comprises means for selecting one or more sperm cells or precursors thereof; means for transducing the one or more selected sperm cells or precursors thereof with at least one RNA transcript or protein; means for combining the transduced sperm cell or precursors thereof with an egg cell for fertilization; and means for receiving information related to the progeny resulting from the fertilization. In an embodiment, the information related to the progeny includes microscopic or molecular information related to testing the progeny as described herein.

In an embodiment, the means for selecting one or more sperm cells or precursors thereof includes one or more of a microarray of at least one gene or protein, hylaronic acid binding assay, or microscopic analysis. In an embodiment, the means for transducing the one or more selected sperm cells or precursors thereof includes one or more of injection, episomal vector, electroporation, or conjugation. In an embodiment, the means for combining the transduced sperm cell or precursors thereof includes a cell culture dish. In an embodiment, the means for receiving molecular information related to the progeny resulting from the fertilization includes one or more of genetic analysis of a polar body, microscopic visualization, fluorescence-activated cell sorting (FACS), or visual tag of a surface or emitted molecule.

In an embodiment, a computer-implemented method comprises circuitry for selecting one or more sperm cells or precursors thereof; circuitry for transducing the one or more selected sperm cells or precursors thereof with at least one RNA transcript or protein; circuitry for combining the transduced sperm cell or precursors thereof with an egg cell for fertilization; and circuitry for receiving molecular information related to the progeny resulting from the fertilization.

In an embodiment, a system for plant breeding, comprises a computing device operable to provide a protocol or execute a protocol to select one or more sperm cells or precursors thereof based on predetermined criteria; transduce the one or more selected sperm cells or precursors thereof with at least one RNA transcript or protein; combining a transduced sperm cell or precursor thereof with an egg cell for fertilization; and receiving molecular information related to the progeny resulting from the fertilization.

In an embodiment, the system further includes selecting an egg cell based on predetermined criteria. In an embodiment, the predetermined criteria for selecting an egg cell include one or more of level of normal morphology, age of donor, presence of at least one genetic marker, overall morphology, chromosomal condition, chromatin condition, or storage conditions of the cell. In an embodiment, the storage conditions of the cell include at least one of duration of storage time, storage temperature, storage size, reproductive cell dilution, or storage solution(s). In an embodiment, the at least one genetic marker includes expression of at least one of SPSB2 or TP5313 gene. In an embodiment, the predetermined criteria for selecting a sperm cell include one or more of level of motility, ability to bind hyaluronic acid, high level of normal morphology, chromatin condition, storage conditions of the cell, or ability to utilize ATP.

In an embodiment, the storage conditions of the cell include at least one of duration of storage time, storage temperature, storage size, reproductive cell dilution, or storage solution(s). In an embodiment, a system, comprises one or more input/output devices having a non-transitory signal bearing medium operable to select one or more sperm cells or precursors thereof; provide a first protocol and/or instruct a computing device to execute the first protocol for transduction of the selected sperm cell or precursor with at least one RNA or protein; provide a second protocol and/or instruct a computing device to execute the second protocol for combining the transduced sperm cell with an egg cell for fertilization. In an embodiment, the system further includes provide a third protocol and/or instruct a computing device to execute the third protocol for analyzing or receiving molecular information related to the progeny resulting from the fertilization.

In an embodiment, a first device is utilized to select one or more sperm cells or precursors thereof. In an embodiment, a second device is utilized to transduce the one or more selected sperm cells or precursors thereof with at least one RNA transcript or protein, or DNA construct. In an embodiment, a third device is utilized to combine the transduced sperm cell or precursors thereof with an egg cell for fertilization. In an embodiment, a first device to select one or more sperm cells or precursors thereof includes a robotic device or computing device. In an embodiment, the second device to transduce the one or more selected sperm cells or precursors thereof with at least one RNA transcript, protein, or DNA construct includes a robotic device or computing device. In an embodiment, the third device to combine the transduced sperm cell or precursors thereof with an egg cell for fertilization includes a robotic device or computing device. In an embodiment, at least one of the first device, second device, or third device is programmable. In an embodiment, at least two of the first device, second device, or third device are the same device.

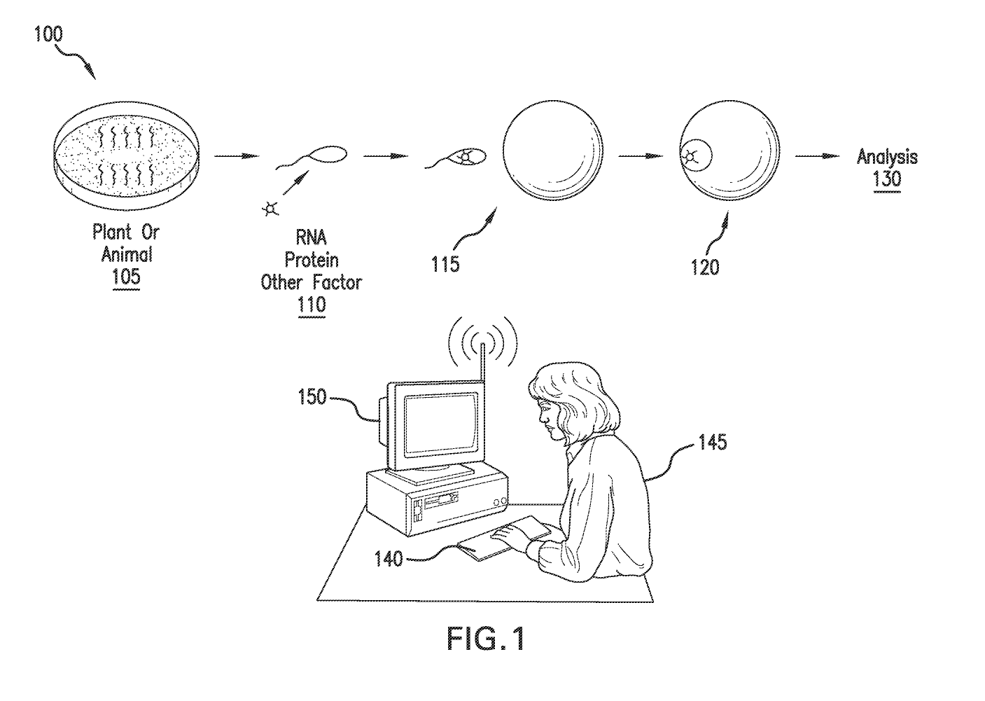

As described in FIG. 1, the method or system 100 includes plant or animal sperm 105 modified with RNA, protein, DNA, or another factor 110, and the modified sperm is utilized for fertilization of an egg 115, resulting in a zygote 120 that includes the modified sperm. Optionally, the progeny (or a polar body) is analyzed 130 at a microscopic or molecular level in order to assess the presence of the factor utilized in modifying the sperm. In an embodiment, an input/output device 140 is utilized for analysis of the progeny or determining particular modifications for the sperm. In an embodiment, an output device (e.g., visual display) is utilized for analysis of the progeny or determining particular modifications for the sperm to a user 145.

As described in the FIGURE, an input/output device 140 operably coupled with a computing device that includes a processing unit, a system memory, and a system bus that couples various system components including the system memory to the processing unit. The system bus may be any of several types of bus structures including a memory bus or memory controller, a peripheral bus, and a local bus using any of a variety of bus architectures. The system bus may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

The system memory includes read-only memory (ROM) and random access memory (RAM). A basic input/output system (BIOS), containing the basic routines that help to transfer information between sub-components within the thin computing device, such as during start-up, is stored in the ROM. A number of program modules may be stored in the ROM or RAM, including an operating system, one or more application programs, other program modules and program data.

A user may enter commands and information into the computing device through input devices, such as a number of switches and buttons, illustrated as hardware buttons, connected to the system via a suitable interface. Input devices may further include a touch-sensitive display with suitable input detection circuitry, illustrated as a display and screen input detector. The output circuitry of the touch-sensitive display is connected to the system bus via a video driver. Other input devices may include a microphone connected through a suitable audio interface, and a physical hardware keyboard (not shown). Output devices may include at least one the display, or a projector display.

In addition to the display, the computing device may include other peripheral output devices, such as at least one speaker. Other external input or output devices, such as a joystick, game pad, satellite dish, scanner or the like may be connected to the processing unit through a USB port and USB port interface, to the system bus. Alternatively, the other external input and output devices may be connected by other interfaces, such as a parallel port, game port or other port. The computing device may further include or be capable of connecting to a flash card memory (not shown) through an appropriate connection port (not shown). The computing device may further include or be capable of connecting with a network through a network port and network interface, and through wireless port and corresponding wireless interface may be provided to facilitate communication with other peripheral devices, including other computers, printers, and so on (not shown). It will be appreciated that the various components and connections shown are examples and other components and means of establishing communication links may be used.

The computing device may be designed to include a user interface. The user interface may include a character, a key-based, or another user data input via the touch sensitive display. The user interface may include using a stylus (not shown). Moreover, the user interface is not limited to an actual touch-sensitive panel arranged for directly receiving input, but may alternatively or in addition respond to another input device such as the microphone. For example, spoken words may be received at the microphone and recognized. Alternatively, the computing device may be designed to include a user interface having a physical keyboard (not shown).

In an embodiment, the system further includes providing an output to at least one user regarding the status of at least one protocol. In an embodiment, the at least one protocol includes one or more of the first protocol, second protocol, or third protocol. In an embodiment, the status of at least one protocol includes beginning, performing, modifying, or completing the protocol. In an embodiment, the status of the protocol includes providing at least one indicator that the protocol has been completed. In an embodiment, the status of the protocol includes providing at least one indicator that the protocol has failed.

In certain instances, one or more components of the computing device may be deemed not necessary and omitted. In other instances, one or more other components may be deemed necessary and added to the computing device.

In certain instances, the computing system typically includes a variety of computer-readable media products. Computer-readable media may include any media that can be accessed by the computing device and include both volatile and nonvolatile media, removable and non-removable media. By way of example, and not of limitation, computer-readable media may include computer storage media. By way of further example, and not of limitation, computer-readable media may include a communication media.

Computer storage media includes volatile and nonvolatile, removable and non-removable media implemented in any method or technology for storage of information such as computer-readable instructions, data structures, program modules, or other data. Computer storage media includes, but is not limited to, random-access memory (RAM), read-only memory (ROM), electrically erasable programmable read-only memory (EEPROM), flash memory, or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by the computing device. In a further embodiment, a computer storage media may include a group of computer storage media devices. In another embodiment, a computer storage media may include an information store. In another embodiment, an information store may include a quantum memory, a photonic quantum memory, or atomic quantum memory. Combinations of any of the above may also be included within the scope of computer-readable media.

Communication media may typically embody computer-readable instructions, data structures, program modules, or other data in a modulated data signal such as a carrier wave or other transport mechanism and include any information delivery media. The term “modulated data signal” means a signal that has one or more of its characteristics set or changed in such a manner as to encode information in the signal. By way of example, and not limitation, communication media include wired media, such as a wired network and a direct-wired connection, and wireless media such as acoustic, RF, optical, and infrared media.

The computing device may also include other removable/non-removable, volatile/nonvolatile computer storage media products. For example, such media includes a non-removable non-volatile memory interface (hard disk interface) reads from and writes for example to non-removable, non-volatile magnetic media, or a removable non-volatile memory interface that, for example, is coupled to a magnetic disk drive that reads from and writes to a removable, non-volatile magnetic disk, or is coupled to an optical disk drive that reads from and writes to a removable, non-volatile optical disk, such as a CD ROM. Other removable/nonremovable, volatile/non-volatile computer storage media that can be used in the example operating environment include, but are not limited to, magnetic tape cassettes, memory cards, flash memory cards, DVDs, digital video tape, solid state RAM, and solid state ROM. The hard disk drive is typically connected to the system bus through a non-removable memory interface, such as the interface, and magnetic disk drive and optical disk drive are typically connected to the system bus by a removable non-volatile memory interface, such as interface.

The drives and their associated computer storage media discussed above provide storage of computer-readable instructions, data structures, program modules, and other data for the computing device.

A user may enter commands and information into the computing device through input devices such as a microphone, keyboard, or pointing device, commonly referred to as a mouse, trackball, or touch pad. Other input devices (not shown) may include at least one of a touch sensitive display, joystick, game pad, satellite dish, and scanner. These and other input devices are often connected to the processing unit through a user input interface that is coupled to the system bus, but may be connected by other interface and bus structures, such as a parallel port, game port, or a universal serial bus (USB).

The computing system may operate in a networked environment using logical connections to one or more remote computers, such as a remote computer. The remote computer may be a personal computer, a server, a router, a network PC, a peer device, or other common network node, and typically includes many or all of the elements described above relative to the computing device, although only a memory storage device. The network logical connections include a local area network (LAN) and a wide area network (WAN), and may also include other networks such as a personal area network (PAN) (not shown). Such networking environments are commonplace in offices, enterprise-wide computer networks, intranets, and the Internet. Included herein is a network for field research station(s).

When used in a networking environment, the computing system is connected to the network through a network interface, such as the network interface, the modem, or the wireless interface. The network may include a LAN network environment, or a WAN network environment, such as the Internet. In a networked environment, program modules depicted relative to the computing device, or portions thereof, may be stored in a remote memory storage device. By way of example, and not limitation, remote application programs as residing on computer medium. It will be appreciated that the network connections shown are examples and other means of establishing communication link between the computers may be used.

In certain instances, one or more elements of the computing device may be deemed not necessary and omitted. In other instances, one or more other components may be deemed necessary and added to the computing device.

The signal generator includes a signal generator configured to generate a signal indicative of the desired characteristic of the plant or plant tissue. In one embodiment, the signal may include a raw data signal, i.e., a capacitance measurement, a change in one or more measurements of a phenotypic characteristic, or an indicator that a desired characteristic has exceeded a (predetermined) threshold. In one embodiment, the signal generator may include a processor circuit, a threshold circuit, an output circuit, or a communications circuit. In one embodiment, the communications circuit may be operable to communicate using an electrical conductor or using a wireless transmission. In one embodiment, the signal generator may include an instance of the thin computing device and the processor circuit may be the processing unit.

In one embodiment, the system actively monitors (e.g., detects, tracks, etc.) a plant or plant tissue located by using at least one of computerized axial tomography, fiber optic thermometry, infrared thermography, magnetic resonance imaging, magnetic resonance spectroscopy, microwave thermography, microwave dielectric spectroscopy, positron emission tomography, ultrasound reflectometry, spectroscopic imaging, visual imaging, infrared imaging, single photon emission computed tomography, global positioning system, satellite imaging, or the like.

### PROPHETIC EXAMPLES

### Prophetic Example 1

### Method to Improve Artificial Insemination Using Sperm Mediated Delivery of Telomerase RNA

A couple having difficulty getting pregnant undergoes artificial insemination. To promote embryogenesis and to increase longevity of the progeny, the donor's sperm are transfected with RNAs for human telomerase prior to insemination. Provision of telomerase to the fertilized oocyte may increase telomere length, promote embryogenesis, and increase lifespan of the resulting child. For example, telomerase gene therapy in mice delays aging and increases longevity, and a strong correlation between short telomeres and mortality risk in humans has been observed, especially at younger ages (see e.g., Lopez-Otin, et al., Cell 153: 1194-1217, 2013 which is incorporated herein by reference). To provide telomerase to the oocyte, telomerase RNAs are transferred into sperm which are then used for artificial insemination.

Sperm are collected and transfected with a messenger RNA (mRNA) encoding human telomerase reverse transcriptase (hTERT) and a telomerase RNA component (TERC). The human telomerase ribonucleoprotein complex including hTERT and TERC is described (see e.g., Meltser, Eukaryon 4: 72-76, 2008; Collins et al., Oncogene 21: 564-569, 2002 and Blackburn et al., Cold Spring Harb. Perspect. Biol. 3: a003558, 2011 which are incorporated herein by reference.) A 4081 nucleotide complementary DNA (cDNA) encoding hTERT (available from OriGene Technologies Inc., Rockville, Md.) and a 451 nucleotide human TERC cDNA (see e.g., National Center for Biotechnology Information Reference Sequence online at: ncbi.nih.gov/nuccore/NR 001566.1, sequence for substance P, neuropeptide gamma, neurokinin A (Oryctolagus cuniculus) are used to produce the corresponding RNAs in vitro using T7 RNA polymerase (kits and reagents to produce RNA's in vitro are available from ProMega Corp., Madison, Wis.).

Alternative methods and materials may be used to produce hTERT mRNA and TERC template RNA with modified nucleosides which are resistant to degradation (see e.g., U.S. Patent Application No. 2012/0065252 by Schrum et al. published on Mar. 15, 2012 which is incorporated herein by reference). For example a complementary DNA (cDNA) sequence encoding hTERT is transcribed in vitro using a mRNA synthesis kit (available from Epicenter Biotechnologies, Madison, Wis.), and modified nucleotides (e.g., pseudouridine and 5-methyl-cytidine available from TriLink Biotech, San Diego, Calif.) are substituted for uridine triphosphate and cytidine triphosphate. The modified, in vitro transcribed RNAs are purified with a RNA purification kit (available from Ambion/Applied Biosystems (Austin, Tex.).

Purified, modified RNAs, hTERT and TERC are transferred into sperm in vitro prior to artificial insemination. To isolate sperm: Semen is obtained by masturbation and immediately diluted in wash buffer to remove seminal fluid components. For example, semen is diluted with a sodium citrate, EDTA, glucose buffer with pH approximately 6.8 and the sperm are recovered by centrifugation at approximately 800×g (see e.g., U.S. Pat. App. Pub. No. 2009/0158450, which is incorporated herein by reference). The wash procedure is repeated and the sperm are re-suspended at a concentration of approximately 1×10⁸ cells/mL in wash buffer at 25° C. To transfer the telomerase RNAs into the sperm, approximately 10⁸ sperm are incubated with 5 μg of hTERT mRNA and 5 μg TERC RNA for 60 minutes at approximately 17° C. in an incubator with 5% CO₂ in air (see e.g., U.S. Pat. App. Pub. No. 2009/0158450 Ibid. and U.S. Pat. App. Pub. No. 2004/0031070; each of which is incorporated herein by reference). Sperm plus telomerase RNAs are incubated for approximately 1 additional minute at 37° C. and used immediately for artificial insemination.

Procedures and materials for ovarian hyper-stimulation and intrauterine insemination may be adapted for particular embodiments (see e.g., Bagis et al., Human Reproduction 25: 1684-1690, 2010 which is incorporated herein by reference). For example, approximately 1 mL of transduced sperm cell suspension (approximately 10⁸ sperm cells) is delivered to the uterine cavity using a 2 mL syringe and a sterile catheter.

Progeny resulting from insemination with sperm carrying telomerase RNAs are analyzed to assess the length of their telomeres. For example fetal cells obtained from maternal blood or from amniotic fluid are analyzed using quantitative polymerase chain reaction and an oligomer reference standard to determine absolute telomere length (see e.g., O'Callaghan and Fenech, Biological Procedures Online 13:3, 2011 available online at doi:10.1186/1480-9222-13-3 which is incorporated herein by reference). The telomere length may be compared to the mean length of telomeres for young and old subjects and for disease conditions (see e.g., O'Callaghan and Fenech Ibid., and Collins and Mitchell, Oncogene 21: 564-579, 2002 which are incorporated herein by reference).

### Prophetic Example 2

### Transfer of mRNA into Sperm of an Infertile Man Prior to Artificial Insemination to Promote Embryogenesis and Implantation

An infertile man with sperm displaying normal morphology and motility is treated by transferring messenger RNAs (mRNAs) into his sperm in vitro prior to artificial insemination of his partner. The transferred mRNAs encode pregnancy-specific β-1-glycoprotein (PSG1) and human leukocyte antigen-E (HLA-E), proteins which are important for implantation and early embryo development (see e.g., Avendaño et al., Human Reproduction 24: 270-277, 2009 which is incorporated herein by reference). Moreover, reduced sperm levels of PSG1 and HLA-E mRNAs are associated with male infertility.

Messenger RNAs for PSG1 and HLA-E are prepared in vitro with fluorescent ribonucleotides which allow detection of the mRNAs following transfer into sperm. Complementary cDNA clones encoding human HLA-E and PSG1 (available from OriGene Technologies, Inc., Rockville, Md.) are transferred to a T7 RNA polymerase vector for in vitro transcription (RNA expression kits and vectors are available from Promega Corp., Madison, Wis.). A fluorescent ribonucleotide is incorporated in the mRNAs to synthesize fluorescent mRNAs which are detected after uptake by sperm. Methods and fluorescent ribonucleotides for synthesis of fluorescent RNA may be adapted for particular embodiments (see e.g., Stengel et al., Anal. Chem. 82(3): 1082. doi:10.1021/ac902456n which is incorporated herein by reference). In vitro transcribed RNAs are purified with a RNA purification kit (available from Ambion/Applied Biosystems (Austin, Tex.) prior to transfer into sperm.

Fluorescent mRNAs encoding PSG1 and HLA-E are transferred into sperm from the infertile male by lipofection. Freshly obtained semen are diluted in a human tubal fluid (HTF) containing human serum albumin (HSA) (available from Irvine Scientific, Santa Ana, Calif.) and the sperm are washed by centrifugation several times to remove seminal fluid components (see e.g., Avendaño et al., Ibid.). Liposomes containing fluorescent PSG1 and HLA-E mRNA are prepared with LipofectAMINE (available from Life Technologies, Grand Island, N.Y.) according to manufacturer's instructions. Approximately 10⁹ sperm in 0.5 mL are combined with 0.5 mls of liposomes containing 10 micrograms of each fluorescent mRNA and the mixture is incubated approximately 2 hours at room temperature (see e.g., Harel-Markowitz et al., Biology of Reproduction 80: 1046-1052, 2009 which is incorporated herein by reference. Following lipofection the sperm that have taken up fluorescent RNA are selected using fluorescence spectroscopy and flow cytometry. Methods to quantitate uptake of fluorescent nucleic acids by sperm may be adapted for particular embodiments (see e.g., De Cecco et al., J. Biomolec. Techniques: 21: 61-65, 2010 which is incorporated herein by reference). The sperm are then sorted on a fluorescence activated cell sorter (FACS) based on their fluorescence due to fluorescent PSG1 and HLA-E mRNA. Methods and instruments to characterize sperm using flow cytometry may be adapted for particular embodiments (see e.g., U.S. Pat. No. 4,559,309 issued to Evenson et al. on Dec. 17, 1985 which is incorporated herein by reference.

Sperm selected for their content of fluorescent mRNA are used for artificial insemination. Procedures and materials for ovarian hyper-stimulation and intrauterine insemination may be adapted for particular embodiments (see e.g., Bagis et al., Human Reproduction 25: 1684-1690, 2010 which is incorporated herein by reference). For example, approximately 1 mL of transduced sperm cell suspension (approximately 10⁸ sperm cells) is delivered to the uterine cavity using a 2 mL syringe and a sterile catheter.

### Prophetic Example 3

### Sperm-Mediated Transfer of c-Myc Protein and Transient Activation of Telomerase

A couple having difficulty getting pregnant undergoes artificial insemination. To promote embryogenesis and to increase longevity of the progeny, the donor's sperm are transfected with a transcription factor, c-Myc, to transiently activate transcription of the human telomerase reverse transcriptase (hTERT) gene in the zygote. The c-Myc protein directly binds to promoter sequences of the hTERT gene and activates transcription of hTERT mRNA which in turn are translated to effect an increase in telomerase enzymatic activity (see e.g., Horikawa and Barrett, Carcinogenesis 24: 1167-1176, 2003 which is incorporated herein by reference). Provision of telomerase to the zygote may increase telomere length, promote embryogenesis, and increase lifespan of the resulting child. For example, telomerase gene therapy in mice delays aging and increases longevity. In humans a strong correlation between short telomeres and mortality risk has been observed, especially at younger ages (see e.g., Lopez-Otin, et al., Cell 153: 1194-1217, 2013 which is incorporated herein by reference). To activate hTERT gene expression in the zygote, c-Myc protein is transferred into sperm and the sperm are used for artificial insemination.

Liposomes are used to transfer c-Myc protein into the sperm resulting in hTERT gene expression, increased telomerase enzymatic activity and telomere lengthening. The c-Myc transcription factor is expressed in vitro using recombinant DNA methods and purified prior to inclusion in liposomes. Expression and isolation of human c-Myc protein is described (see e.g., Miyamoto et al., Proc. Natl. Acad. Sci. USA 82: 7232-7236, 1985 which is incorporated herein by reference). Antibodies specific for c-Myc may be used to purify c-Myc protein for loading liposomes. Liposomes are formed using a method that mixes phospholipids (e.g., phosphoethanolamine, phosphocholine) and a detergent (e.g., sodium cholate at a concentration of 9-15 mM) and c-Myc protein (e.g., approximately 10 μg/mL) to form a mixed micelle solution. The mixture is dialyzed versus a saline solution over approximately 14 hours to remove the detergent and yield unilamellar vesicles that encapsulate c-Myc protein. Methods and materials to form liposomes suitable for fusion with sperm and delivery of macromolecules may be adapted for particular embodiments (see e.g., Garrett et al., Biochimica et Biophysica Acta 1417: 77-88, 1999 which is incorporated herein by reference).

Freshly obtained semen are diluted in a human tubal fluid (HTF) (available from Irvine Scientific, Santa Ana, Calif.) and the sperm are washed by centrifugation several times to remove seminal fluid components (see e.g., Avendaño et al., Human Reproduction 24: 270-277, 2009 which is incorporated herein by reference). Approximately 10⁹ sperm in 0.5 mL are combined with 0.5 mL of liposomes containing approximately 5.0 μg of c-Myc protein and the mixture is incubated approximately 2 hours at room temperature (see e.g., Harel-Markowitz et al., Biology of Reproduction 80: 1046-1052, 2009 which is incorporated herein by reference). Sperm with internalized c-Myc protein may be selected for motility in a swim-up separation procedure. Briefly, sperm are resuspended in HTF supplemented with 3 mg/ml human serum albumin (HSA; available from Irvine Scientific, Santa, Ana, Calif.) and then centrifuged at 300×g. Supernatant HTF is removed and approximately 1 ml of HTF plus HSA is layered over the pellet followed by incubation for 1 hour at 37° C. to allow motile sperm to swim-up. The top layer is removed to isolate the motile sperm (see e.g., Avendaño et al., Ibid.) which are used for artificial insemination.

Procedures and materials for ovarian hyper-stimulation and intrauterine insemination may be adapted for particular embodiments (see e.g., Bagis et al., Human Reproduction 25: 1684-1690, 2010 which is incorporated herein by reference). For example, approximately 1 mL of lipofected sperm cell suspension (approximately 10⁸ sperm cells) is delivered to the uterine cavity using a 2 mL syringe and a sterile catheter.

Progeny resulting from insemination with sperm carrying c-Myc protein are analyzed to assess the length of their telomeres. For example fetal cells obtained from maternal blood or from amniotic fluid are analyzed using quantitative polymerase chain reaction and an oligomer reference standard to determine absolute telomere length (see e.g., O'Callaghan and Fenech, Biological Procedures Online 13:3, 2011 available online at doi:10.1186/1480-9222-13-3 which is incorporated herein by reference). The telomere lengths may be compared to the mean length of telomeres for young and old subjects and for disease conditions (see e.g., O'Callaghan and Fenech Ibid., and Collins and Mitchell, Oncogene 21: 564-579, 2002 which are incorporated herein by reference).

### Prophetic Example 4

### Transfer of Essential Messenger RNA into Pollen Cells to Optimize Plant Fertilization and Zygote Development Following Artificial Pollination

Cross-pollination is used to create hybrid plants for agriculture, and artificial pollination methods are used to implement plant breeding schemes and to allow manipulation of the pollen prior to fertilization. However, artificial pollination is often not as efficient as natural pollination especially in some plant species (see e.g., Rai, Nagendra; Rai, Mathura (2006) Heterosis Breeding in Vegetable Crops, New India Publishing, New Delhi, ISBN 978-81-89422-03-5). To improve the efficiency of artificial pollination, messenger RNA (mRNA) encoding proteins essential for fertilization and zygote development are transferred into sperm cells in mature pollen grains.

To improve pollination efficiency maize pollen are transformed with mRNAs to promote fertilization and zygote development. For example, pollen are transformed with mRNA encoding LAT52, a cysteine-rich protein important for fertilization, and AtTMPK (thymidylate kinase) an enzyme essential for zygote development (see e.g., Muschietti et al., The Plant Journal 6: 321-328, 1994 and Ronceret et al., The Plant Journal 53: 776-789, 2008 which are incorporated herein by reference). LAT52 and AtTMPK mRNA are synthesized with modified nucleosides which are resistant to degradation (see e.g., U.S. Pat. App. Pub. No. 2012/0065252, which is incorporated herein by reference). Complementary DNA (cDNA) sequences encoding LAT52 and AtTMPK are transcribed in vitro using a mRNA synthesis kit (available from Epicenter Biotechnologies, Madison, Wis.), and modified nucleotides (e.g., pseudouridine and 5-methyl-cytidine available from TriLink Biotech, San Diego, Calif.) are substituted for uridine triphosphate and cytidine triphosphate. The modified, in vitro transcribed RNAs are purified with a RNA purification kit (available from Ambion/Applied Biosystems (Austin, Tex.) and the purified mRNAs are transformed into maize pollen.

Maize pollen is transformed with LAT52 and AtTMPK mRNA prior to artificial pollination. For example, 0.1 grams of pollen grains are collected during anthesis and dispersed in 0.3 M sucrose solution and 0.2 mg/ml of modified, nuclease-resistant LAT52 and AtTMPK mRNA are added to the pollen. The suspension is sonicated at 300 watts for 4 intervals of 10 seconds each. Methods to transfer nucleic acids into pollen using sonication may be adapted for particular embodiments (see e.g., Eapen, Physiol. Mol. Biol. Plants 17: 1-8, 2011 which is incorporated herein by reference). The pollen is used to pollinate the stigma of emasculated or male sterile flowers. Pollen transformed with LAT52 and AtTMPK mRNA may be used to create hybrids with improved phenotypes that cross pollinate and fertilize at low efficiency. See for example cross hybrids described in Rai, Nagendra; Rai, Mathura (2006) Heterosis Breeding in Vegetable Crops, New India Publishing, New Delhi, ISBN 978-81-89422-03-5.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system comprising:
a computing device operable to at least one of
provide a protocol or execute a protocol to
select one or more sperm cells or precursors thereof based on predetermined criteria;
transduce the one or more selected sperm cells or precursors thereof with at least one RNA transcript or protein;
combine a transduced sperm cell or precursor thereof with an egg cell for fertilization; and
receive molecular information related to the progeny resulting from the fertilization.

2. The system of claim 1, further including selecting an egg cell based on predetermined criteria.

3. The system of claim 2, wherein the predetermined criteria for selecting an egg cell include one or more of level of normal morphology, age of donor, presence of at least one genetic marker, overall morphology, chromosomal condition, chromatin condition, or storage conditions of the cell.

4. The system of claim 3, wherein the storage conditions of the cell include at least one of duration of storage time, storage temperature, storage size, reproductive cell dilution, or storage solution(s).

5. The system of claim 3, wherein the at least one genetic marker includes expression of at least one of SPSB2 or TP5313 gene.

6. The system of claim 1, further including selecting a sperm cell based on predetermined criteria.

7. The system of claim 6, wherein the predetermined criteria for selecting a sperm cell include one or more of level of motility, ability to bind hyaluronic acid, high level of normal morphology, chromatin condition, storage conditions of the cell, or ability to utilize ATP.

8. The system of claim 7, wherein the storage conditions of the cell include at least one of duration of storage time, storage temperature, storage size, reproductive cell dilution, or storage solution(s).

9. The system of claim 1, wherein the progeny includes at least one of a zygote, morula, blastula, embryo, fetus, neonate, child, adult, seed, or plant stage progeny.

10. The system of claim 1, wherein the sperm cell precursor includes at least one of a spermatogonium, spermatocyte, or spermatid.

11. The system of claim 1, wherein the egg cell includes at least one oocyte, ootid, or oogonium.

12. The system of claim 1, wherein receiving molecular information related to the progeny includes receiving molecular information regarding one or more of genetic sequencing, RNA sequencing, or protein sequencing.

13. The system of claim 1, wherein receiving molecular information related to the progeny includes receiving molecular information regarding the quality of at least one gene or gene product.

14. The system of claim 13, wherein the quality of at least one gene or gene product includes information regarding at least one of telomere length, protein folding, gene expression, nucleotide or amino acid deletion, nucleotide or amino acid insertion, nucleotide or amino acid mutation, protein mutation, methylation status, or demethylation status.

15. The system of claim 1, further including identifying at least one desired genetic, transcription, or translation characteristic from the sequence of the progeny.

16. The system of claim 1, further including at least one of providing or executing a protocol for implanting the progeny into a suitable subject under conditions sufficient for the progeny to develop.

17. The system of claim 16, wherein the subject includes at least one animal.

18. The system of claim 17, wherein the animal includes a reptile, amphibian, bird, mammal or fish.

19. The system of claim 16, further including receiving a second round of molecular information related to the progeny following implantation into the suitable subject.

20. The system of claim 16, wherein the subject includes at least one plant.

21. The system of claim 1, wherein receiving molecular information related to the progeny includes receiving information regarding at least a portion of at least one cell from the progeny.

22. The system of claim 1, wherein one or more RNA transcripts include telomerase RNA.

23. The system of claim 1, further including at least one of providing or executing a protocol to determine whether the RNA transcripts are translated in the progeny.

24. The system of claim 23, wherein the translation is transient.

25. The system of claim 1, further including providing an output to at least one user regarding the status of the protocol.

26. The system of claim 25, wherein the status of the protocol includes beginning, performing, modifying, or completing the protocol.

27. The system of claim 25, wherein the status of the protocol includes providing at least one indicator that the protocol has been completed.

28. The system of claim 27, wherein the status of the protocol includes providing at least one indicator that the protocol has failed.

29. A system, comprising:
one or more input/output devices having a non-transitory signal bearing medium operable to
select one or more sperm cells or precursors thereof;
provide a first protocol and/or instruct a computing device to execute a first protocol for transduction of the selected sperm cell or precursor with at least one DNA, RNA or protein;
provide a second protocol and/or instruct a computing device to execute a second protocol for combining the transduced sperm cell with an egg cell for fertilization.

30. The system of claim 29, further including
provide a third protocol and/or instruct a computing device to execute a third protocol for receiving molecular information related to the progeny resulting from the fertilization.

31. A computer-implemented method comprising:
means for selecting or instructing a first device to select one or more sperm cells or precursors thereof;
means for transducing or instructing a second device to transduce the one or more selected sperm cells or precursors thereof with at least one RNA transcript or protein;
means for combining or instructing a third device to combine the transduced sperm cell or precursors thereof with an egg cell for fertilization; and
means for receiving molecular information related to the progeny resulting from the fertilization.

32. The method of claim 31, wherein the means for selecting or instructing a device to select one or more sperm cells or precursors thereof includes one or more of a microarray of at least one gene or protein, hylaronic acid binding assay, or microscopic analysis.

33. The method of claim 31, wherein the means for transducing or instructing a device to transduce the one or more selected sperm cells or precursors thereof includes one or more of injection, episomal vector, liposome, electroporation, or conjugation.

34. The method of claim 31, wherein the means for combining or instructing a device to combine the transduced sperm cell or precursors thereof includes a cell culture dish.

35. The method of claim 31, wherein the means for receiving molecular information related to the progeny resulting from the fertilization includes one or more of genetic analysis of a polar body, microscopic visualization, fluorescence-activated cell sorting (FACS), or visual tag of a surface or emitted molecule.

36. The method of claim 31, wherein the first device to select one or more sperm cells or precursors thereof includes a robotic device or computing device.

37. The method of claim 31, wherein the second device to transduce the one or more selected sperm cells or precursors thereof with at least one RNA transcript or protein includes a robotic device or computing device.

38. The method of claim 31, wherein the third device to combine the transduced sperm cell or precursors thereof with an egg cell for fertilization includes a robotic device or computing device.

39. The method of claim 31, wherein at least one of the first device, second device, or third device is programmable.

40. The method of claim 31, wherein at least two of the first device, second device, or third device are the same device.

41. A computer-implemented method comprising:
circuitry for selecting one or more sperm cells or precursors thereof;
circuitry for transducing or instructing a first device to transduce the one or more selected sperm cells or precursors thereof with at least one RNA transcript or protein;
circuitry for combining or instructing a second device to combine the transduced sperm cell or precursors thereof with an egg cell for fertilization; and
circuitry for receiving molecular information related to the progeny resulting from the fertilization.

42. The method of claim 41, wherein the first device to select one or more sperm cells or precursors thereof includes a robotic device or computing device.

43. The method of claim 41, wherein the second device to transduce the one or more selected sperm cells or precursors thereof with at least one RNA transcript or protein includes a robotic device or computing device.

44. The method of claim 41, wherein at least one of the first device or second device is programmable.

45. The method of claim 41, wherein the first device and second device are the same device.
